(19) United States
(12) Patent Application Publication
(10) Pub. No.: US 2009/0306339 A1
(43) Pub. Date: Dec. 10, 2009

(54) Preparation of Fine Particles

(76) Inventor: Huibert Albertus Van Boxtel, Holland (NL)

Correspondence Address: MORGAN LEWIS & BOCKIUS LLP, 1111 PENNSYLVANIA AVENUE NW, WASHINGTON, DC, 20004, US

(21) Appl. No.: 12/441,943

(22) PCT Filed: Jul. 20, 2007
(86) PCT No.: PCT/GB2007/002786

§ 371 Date: Mar. 19, 2009

(30) Foreign Application Priority Data
Sep. 19, 2006 (EP) 06120915.1

(51) Int. Cl. A61K 38/13; C07D 305/14; C07C 69/76

(52) U.S. Cl. 530/321; 549/510; 560/52

(57) ABSTRACT

A process for the precipitation of an organic compound comprising mixing simultaneously introduced streams of a solution and a precipitation agent in a chamber using a mechanical stirrer in the presence of an amphiphilic polymer. The process may be operated in a continuous manner and is particularly useful for providing low solubility organic compounds (e.g. pharmaceuticals) in readily dispersible, nano-sized particulate form up to manufacturing scale.

This invention relates to a process for the precipitation of organic compounds in a fine particulate form.

In the pharmaceuticais field, there are many factors which can affect the bioavailability of drugs and therefore their effectiveness at treating diseases and medical disorders. These factors include the particle size, the particle size distribution and the dissolution rate of the active ingredient. Poor bioavailability is a significant problem encountered in the development of pharmaceutical compositions, particularly those containing an active ingredient that is poorly soluble in water. Poorly water-soluble drugs, e.g., those having a solubility less than about 10 mg/ml, tend to be eliminated from the gastrointestinal tract before being absorbed into the circulation. Moreover, poorly water soluble drugs can give rise to difficulties when required for intravenous administration in terms of blocking needles and even blocking tiny blood vessels in patients.

It is known that the rate of dissolution of particulate drugs can increase with increasing surface area, e.g. by decreasing particle size. Consequently, methods of making finely divided drugs have been studied and efforts have been made to control the size and size range of drug particles in pharmaceutical compositions. For example, dry milling techniques have been used to reduce particle size and hence influence drug absorption. However, in conventional dry milling, the limit of fineness is often in the region of 100 microns (100,000 nm) when material begins to cake on the walls of the milling chamber. Wet grinding is beneficial in further reducing particle size, but flocculation restricts the lower particle size limit in many cases to approximately 10 microns (10,000 nm).

Commercial airjet milling techniques have provided particles ranging in average particle size from as low as about 1 micron up to about 50 microns (1,000to 50,000 nm).

One known method for preparing small particles of organic compounds makes use of solvents, anti-solvents and impinging jets, as disclosed in Chapter 18 of. Johnson, Brian K.; Saad, Walid; Prud'homme, Robert K. Department of Chemical Engineering, Princeton University, Princeton, N.J., USA. ACS Symposium Series (2006), 924(Polymeric Drug Delivery II), 278-291. Publisher: American Chemical Society, CODEN; ACSMC8 ISSN: 0097-6156. The impinging jet method generally comprises providing two substantially diametrically opposed jet streams of solvent and anti-solvent that impinge to create an immediate high turbulence impact. The anti-solvent causes any compounds present in the solvent to precipitate out of solution, thereby giving a particulate precipitate.

In our experience the opposed impinging jet method presents certain practical difficulties. Accurate positioning and alignment of the jet nozzles is required because if the jets are slightly out of line the solvent and anti-solvent do not mix thoroughly and a wide particle size distribution can result. Furthermore, even small deviations in the orientation of the jet nozzles can cause a precipitate to form on a nozzle which can then block it. Insufficient flow rates from one or more of the jet nozzles may affect the quality of the entire batch being produced, especially if a majority of the solutions are not micro mixed at the desired point of impact. In such a case a narrow, small size particle distribution cannot be achieved. Generally, the preferred flow for the impinging jet streams has little room for variance.

Gaβmann et al (Eur. J. Pharm. Biopharm. 40(2)64-72 (1994)) prepared hydrosols comprising drug actives on the laboratory scale. They injected a solution of the drug (which had low water-solubility) dissolved in an organic solvent into an open beaker already containing water and a stabilising agent with stirring. The stabilising agents included chemically modified gelatines, Poloxamer™ 188 (a block copolymer stated as having a molecular weight of 8,400) and Poloxamer™ 407 (a block copolymer stated as having a molecular weight of 12,500). Gaβmann et al commented that their process is almost impossible to scale-up. Gaβmann et al also prepared hydrosols using a static mixer relying on turbulent flow for the mixing. The inlets and outlet shared the same axis of flow and the glass tube through which they passed contained baffles to create turbulence.

U.S. Pat. No. 4,826,689 describes a method for making particles of water-insoluble drugs comprising the slow infusion of water into a solution of the drug in an organic solvent. The water, which acts as an anti-solvent, may contain a surfactant, e.g. Pluronic F-68 or a gelatine. This batch-wise process appears to be quite slow and laborious.

U.S. patent application publication No. 2005/0202095 A1 describes an alternative process for making fine particles by mixing an anti-solvent and a solvent containing the desired compound in an off-the-shelf rotor stator device such as a Silverson Model L4RT-A Rotor-Stator. However the resultant particles were very large, e.g. in the Examples the precipitated glycine particles ranged in size from 4,4 microns to 300 microns.

U.S. Pat. No. 5,543,158 describes the preparation of injectable nanoparticles having poly(alkylene glycol) (“PEG”) chains on the surface comprising a biodegradable solid core containing a biologically active ingredient. These nanoparticles may contain amphiphilic copolymers comprising PEG and were prepared in a batch wise manner by vortexing and sonicating oil-in-water emulsions for 30 seconds, followed by slow evaporation of organic solvent by gentle stirring for several hours. The process was therefore rather time consuming and laborious.

U.S. Pat. No. 7,153,520 describes the preparation of implants for the sustained delivery of drugs comprising an amphiphilic diblock copolymer and a poorly water-soluble drug contained in an implant made largely of a biodegradable polymer. The compositions are prepared by simply mixing various components contained in a round-bottom flask.

There exists a need for a process for preparing organic compounds, particularly pharmaceutical actives, with a small particle size without the need for potentially wasteful and damaging milling and without the need for accurately positioned jets which might clog. Ideally the process is operable on the industrial scale, is rapid, not unduly complicated and leads to particles which can rapidly be redispersed.

According to the present invention there is provided a process for the precipitation of an organic compound, wherein:
(a) a solution (I) of the organic compound in a solvent is introduced via a first inlet into a mixing chamber,
(b) a precipitation agent (It) is introduced, simultaneously with step (a), via a second inlet into the mixing chamber;
(c) the solution (I) of the organic compound and the precipitation agent (II) are mixed thereby forming a precipitate of the organic compound and a liquid phase; and
(d) the precipitate of the organic compound and the liquid phase is discharged from the chamber via one or more outlets;
   wherein step (c) is performed using a mechanical stirring means in the presence of an amphiphilic polymer.

In this document (including its claims), the verb “comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

In this document, the term “organic compounds” in its broadest sense refers to compounds comprising at least one carbon atom. Usually, organic compounds also comprise hydrogen atoms. Very often organic compounds also comprise hetero-atoms, e.g. oxygen atoms, nitrogen atoms, and/or sulphur atoms. In particular the term “organic compounds” refers what is normally considered an organic compound in the field of pharmaceutical, dye, agricultural and chemical industry. The term “organic compounds” also include compounds that comprise a metal atom, i.e. organometallic compounds such as haemoglobin, and salts. The term “organic compounds” includes “biological” organic compounds such as hormones, proteins, peptides, carbohydrates, amino acids, lipids, vitamins, enzymes and the like. The term “organic compounds” also encompasses different crystalline forms, i.e. polymorphs, hydrates and solvates, as well as salts including addition salts.

The term “precipitation” refers to a subclass of the field of solution precipitation. Precipitation is often recognised by one or more of the following characteristics: (i) low solubility of the precipitated particles, (ii) fast process, (Hi) small particle size and (iv) irreversibility of the process (W. Gerhartz in: Ullman's encyclopaedia of Industrial Chemistry, vol. B2 5^{th} ed., VHC Verlagsgesselfschaft mbH, Weinheim, FGR, 1988). In the context of this invention, a suitable definition for precipitation is the relatively rapid formation of a sparingly soluble solid phase from a liquid solution phase (Handbook of industrial crystallization, Edited by Allan S. Myerson, Butterworth Heinemann, Oxford, p141).

Generally two types of processes resulting in precipitation can be discerned:
a first type of process is anti-solvent (also referred to as anti-solvent and non-solvent) precipitation. A dissolved organic compound is mixed with a solvent that lowers its solubility so that a precipitate will form. A modification of the anti-solvent precipitation is that a dissolved organic compound is not necessarily mixed with an anti-solvent but is mixed in such way that the solubility of the precipitating solvent is lowered such that nuclei are formed. This can be realised by variations in for example temperature, pH (addition of acid or alkaline solutions), ionic strength and the like and combinations of such factors.
a second type of process is reaction precipitation. Two components are mixed resulting in the formation of a newly formed organic compound and due to the low solubility of the formed organic compound under the used mixing or reaction conditions a precipitate will form.

Obviously, the term “precipitation” encompasses any process wherein small solid particles are formed, e.g. including but not limited to crystallisation.

The term “anti-solvent” or “non-solvent” is normally to be understood as a liquid in which the solubility of the organic compound is less than 1% by weight, more preferably less than 10⁻²% by weight, based on the total weight of the solvent and the organic compound, at a temperature of 20° C. and a pressure of 1 bar. The solvent may be polar or apolar. The solvent may be protic or aprotic. The solvent may further be non-ionic or ionic. Preferably however the solvent is or comprises an organic solvent. Preferably the solvent and the anti-solvent are miscible.

With the term “supersaturation” is meant a concentration of an organic compound that is in excess of saturation under the given conditions, i.e. solvent or solvent mixture, temperature, pH, ionic strength etc.

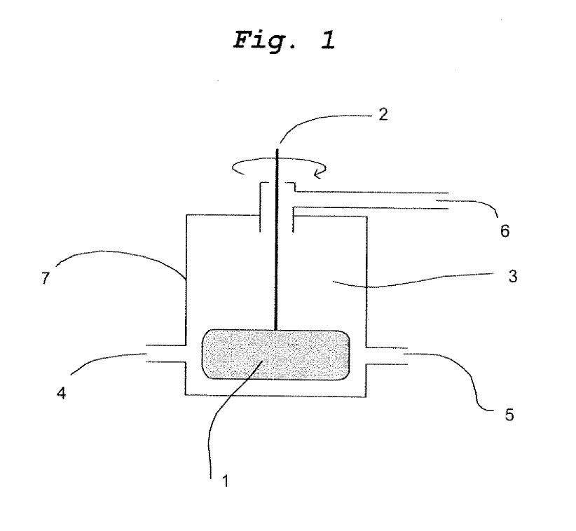

FIG. 1 shows a general representation of a device which may be used to perform the process of the present invention.

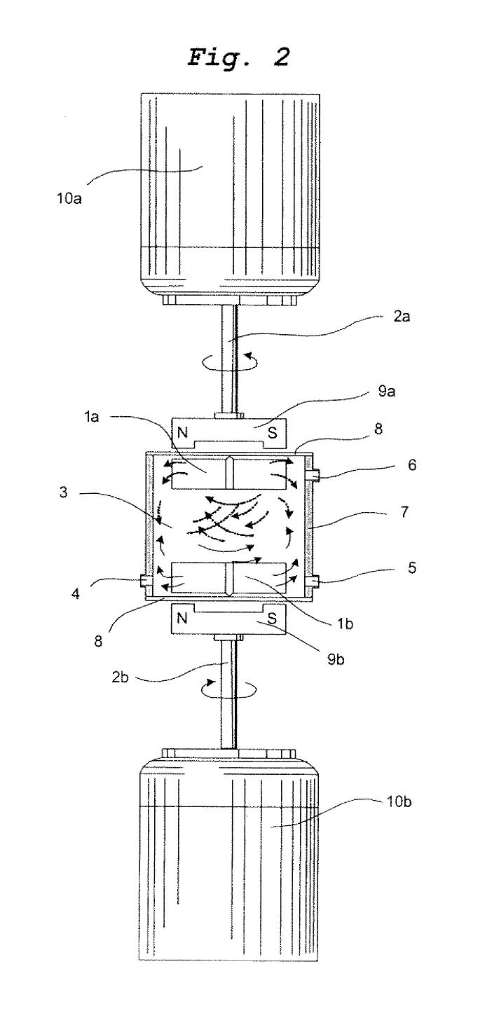

FIG. 2 shows a cross-sectional view of a preferred embodiment of the device.

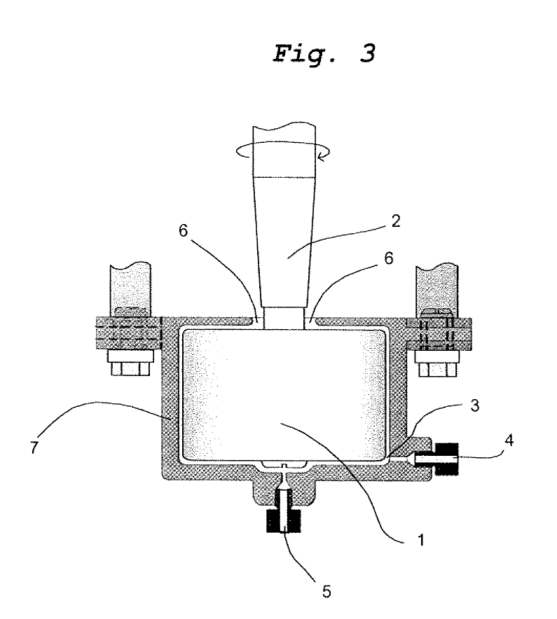

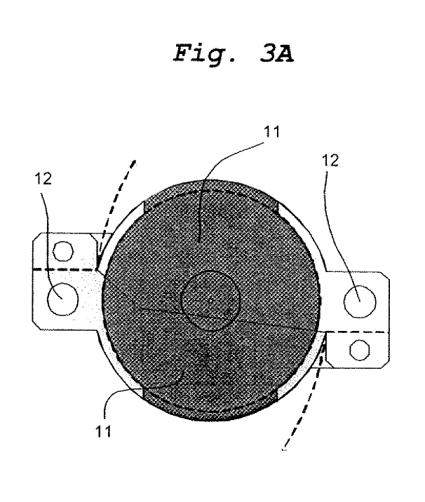

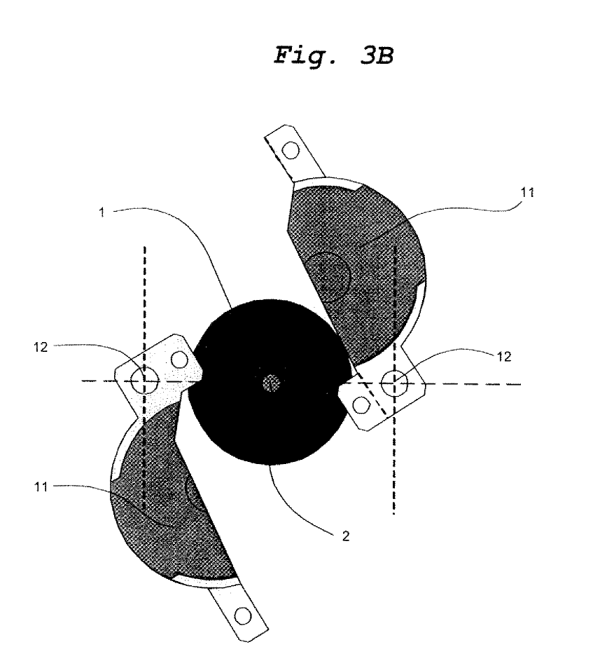

FIG. 3 shows a cross-sectional view of another preferred embodiment of the device.

FIGS. 3A and 3B show top views of a more preferred embodiment of the device shown in FIG. 3.

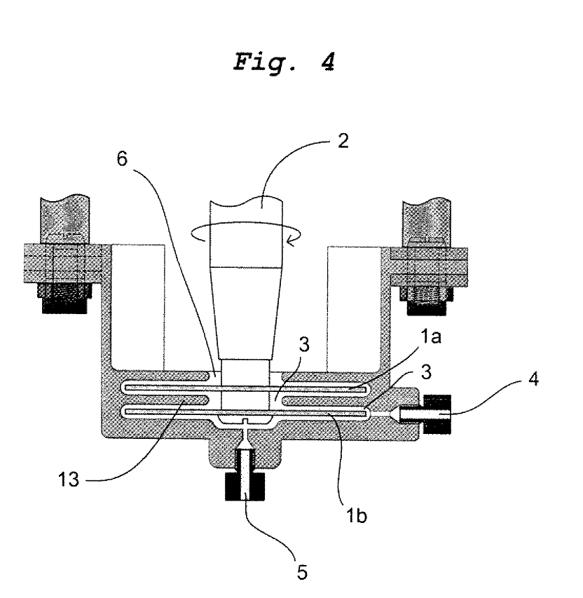

FIG. 4 shows a cross-sectional view of yet another preferred embodiment of the device.

### Key to the Symbols Used in the Drawings:

1, 1a, 1b: Mechanical stirring means
2, 2a, 2b: Axis or shaft
3: Mixing chamber
4: First inlet for feeding a solution (I)
5: Second inlet for feeding a precipitating agent (II)
6: Outlet
7: Mixing chamber wall
8: Seal plate
9a, 9b: Outer magnet
10a, 10b: Motors
11: Moveable chamber part
12: Hinge

13: Separating wall.

In a typical process according to the present invention, a solution (I) of the organic compound or a precursor of the organic compound in a solvent is provided which may be fed with a continuous flow via a first inlet into the mixing chamber. Simultaneously, a precipitation-agent (II) may be fed, also with a continuous flow, via a second inlet into the mixing chamber. The mixing chamber may be provided with more than one first inlet for this solution (I) and more than one second inlet for this precipitation agent (II). In a next step, the solution (I) and the precipitation agent (II) are mixed and said mixture provides a supersaturation. Finally, the mixture of the precipitate and the liquid phase is discharged from the mixing chamber, preferably also with a continuous flow, and preferably into a collecting (or receiving) vessel. According to the invention, it is preferred that there is basically no supersaturation at the outlet of the mixing chamber. There may be one outlet or more than one outlet. Additionally, in one embodiment, there are no other openings in the mixing chamber besides the inlets and the outlet(s). This means that no solvents, liquids, solutions, particles and the like can enter or exit the mixing chamber except via the first and second inlets and the outlet. Such chambers are often referred to as “closed type” mixing chambers.

The mixing chamber preferably comprises two inlets and one outlet.

The solution (I) of the organic compound may comprise a single solvent or a mixture of solvents, wherein the solvent or solvents may be polar or apolar, protic or aprotic, and/or non-ionic or ionic. The solvent may also be a gas in the supercritical state, e.g. supercritical carbon dioxide, if that is appropriate.

The preferred nature and composition of the precipitation agent (II) is dependent on the organic compound and the process used and can for example be a solution having a lower temperature (in case of low temperature precipitation), different ionic strength or different pH than the solution (I). The precipitation agent (II) can also be a non-solvent, a mixture of non-solvents, or a mixture of a non-solvent and a solvent.

The process according to the present invention is very suitable for the preparation of very small particles with a narrow average particle size distribution in the lower micron, or even nanometre range. A disadvantage of such small particles is that these tend to be unstable; therefore one or more amphiphilic polymer is included as a stabilisation agent to prevent or slow down particle size growth and agglomeration.

It is preferred that the solution (I) and/or the precipitation agent (II) comprises a wetting agent.

The amphiphilic polymers preferably have an affinity for both the organic compound and water. When the organic compound has a low solubility in water, the amphiphilic polymer will generally possess a hydrophilic part which has an affinity for water and a less hydrophilic part, e.g. a relatively hydrophobic part, which has an affinity for the organic compound. The relatively hydrophilic part of the amphiphilic polymers are often non-ionic (e.g. polyethylene oxide units) and/or ionic (e.g. they have anionic or cationically charged groups) while the less hydrophilic or hydrophobic parts are often electrically neutral and relatively non-polar (e.g. polylactide groups).

Preferred amphiphilic polymers are amphiphilic block copolymers, especially biocompatible amphiphilic block copolymers. The preferable block-type and block-lengths can vary depending on the organic compound to be precipitated and on the preferred average particle size after precipitation. Preferably the amphiphilic polymer comprises hydrophilic and relatively hydrophobic segments. Preferably the amphiphilic polymers are triblock and diblock copolymers, especially diblock copolymers. Typically such copolymers comprise at least one hydrophobic block and at least one hydrophilic block.

Preferred hydrophilic blocks are poly(ethylene glycol) (“PEG”) and/or poly(ethylene glycol) monoether (“PEG ether”) blocks. The preferred ethers have from 1 to 4 carbon atoms, with methyl ether being most preferred. Preferred blocks which are relatively hydrophobic are poly (lactic-co-glycolic)acid (“PLGA”), poly(styrene), poly(butyl acrylate), poly(ε-caprolactone) and especially polylactide (“PLA”) blocks, Polylactides are polyesters formed from the polymerisation of lactic acid. Polylactides exist as poly-L-lactide, poly-D-lactide and poly D,L-lactide.

Preferred biocompatible amphiphilic block copolymers include copolymers comprising one or more PEG and/or PEG ether blocks and one or more polylactide (“PLA”) blocks. Polylactides are polyesters formed from the polymerisation of lactic acid. Polylactides exist as poly-L-lactide, poly-D-lactide and poly D,L-lactide.

Preferably the PEG and PEG ether block(s) have an Mₙ (Mn means the number average molecular weight) of 250 to 5000, more preferably 400 to 4000, especially 500 to 2000, more especially 600 to 1500. Very good results were obtained with a PEG having an Mn of 750. Thus in a preferred process according to the invention the amphiphilic copolymer is an amphiphilic block copolymer comprising a PEG Mₙ 250-5000 block and/or a PEG Mₙ 250-5000 (C₁₋₄-alkyl) ether block, with the preferred Mn of such block(s) being 400 to 4000, especially 500 to 2000, more especially 600 to 1500, and particularly 750. Preferably the PLA block(s) have an Mₙ 250 to 5000, more preferably 400 to 4000, especially 500 to 2000 and more especially from 600 to 1500. Very good results were obtained with a PLA block having an Mn of 1000. A particularly preferred amphiphilic block copolymer is a diblock copolymer of a PEG ether and a PLA having the Mₙs mentioned above, with the preferences for Mₙ in each block being as mentioned above.

Examples of these block copolymers are:
poly(ethylene glycol)-block-polylactide (C₁₋₄-alkyl) ether, PEG Mₙ 350-1500, PLA Mₙ 500-2000;
poly(ethylene glycol)-block-polylactide (C₁₋₄-alkyl) ether, PEG Mₙ 500-1100, PLA Mₙ 600-1600;
poly(ethylene glycol)-block-polylactide (C₁₋₄-alkyl) ether, PEG Mn 600-900, PLA Mₙ 800-1200;
poly(ethylene glycol)-block-polylactide (C₁₋₄-alkyl) ether, PEG Mₙ 700-900, PLA Mₙ 800-1200;
polyethylene glycol)-block-polylactide methyl ether, PEG Mₙ 700-900, PLA Mₙ 800-1200;
poly(ethylene glycol)-block-polylactide (C₁₋₄-alkyl) ether, PEG Mₙ 750, PLA Mₙ 1000; and
poly(ethylene glycol)-block-polylactide methyl ether, PEG Mₙ 750, PLA Mₙ 1000.

Examples of amphiphilic block copolymers include: poly(ethylene glycol)-block-polylactide methyl ether, PEG Mₙ 750, PLA Mₙ 1000 (also known as PEG mono methyl ether Mn 750 PLA Mn 1000);

poly(ethylene glycol)-block-polylactide methyl ether, PEG Mₙ 350, PLA Mₙ 1000;
poly(ethylene glycol)-block-poly(lactone) methyl ether, PEG Mₙ 5000, polylactide Mₙ-5000;
poly(ethylene glycol)-block-poly(ε-caprolactone) methyl ether, PEG Mₙ 5,000, polycaprolactone Mₙ 5,000;
poly(ethylene glycol)-block-poly(ε-caprolactone) methyl ether, PEG Mₙ 5,000, polycaprolactone Mₙ 13,000; and
poly(ethylene glycol)-block-poly(ε-caprolactone) methyl ether, PEG Mₙ 5,000,
polycaprolactone Mₙ 32,000; all of which are commercially available from Sigma-Aldrich Co.

As will be readily understood by those skilled in the art, “methyl ether” refers to a methyl group on one end of the PEG chain (not both ends because this would prevent the PLA from attaching to the PEG). Also the Mn values for the PEG, such in “PEG mono methyl ether Mn 750” refer to the Mn of the PEG per se, not including the extra CH₂ group of the methyl group.

Amphiphilic polymers are available from commercial sources or they may be synthesised ad hoc for use in the process. The amphiphilic polymer may be a single amphiphilic polymer or a mixture comprising two or more (e.g. 2 to 5) amphiphilic polymers. The preparation of the preferred amphiphilic diblock copolymers with poly(alkylene glycol) (PAG) blocks (e.g. poly(ethylene glycol) (PEG) blocks) can be performed in a number of ways. Methods include: (i) reacting a hydrophobic polymer with methoxy poly(alkylene glycol), e.g. methoxy PEG or PEG protected with another oxygen protecting group (such that one terminal hydroxyl group is protected and the other is free to react with the hydrophobic polymer); or (ii) polymerizing the hydrophobic polymer onto methoxy or otherwise monoprotected PAG, such as monoprotected PEG. Several publications teach how to carry out the latter type of reaction. Multiblock polymers have been prepared by bulk copolymerization of D,L-lactide and PEG at 170°-200° C. (X. M. Deng, et al., J. of Polymer Science: Part C: Polymer Letters, 28, 411-416 (1990). Three and four arm star PEG-PLA copolymers have been made by polymerization of lactide onto star PEG at 160° C. in the presence of stannous octoate as initiator, K, J. Zhu, et al., J. Polym. Sci., Polym. Lett, Ed., 24,331 (1986), “Preparation, characterization and properties of polylactide (PLA)-poly(ethylene glycol) (PEG) copolymers: a potential drug carrier”. Triblock copolymers of PLA-PEG-PLA have been synthesized by ring opening polymerization at 180°-190° C. from D,L-lactide in the presence of PEG containing two end hydroxy! groups using stannous octoate as catalyst, without the use of solvent. The polydispersity (ratio Mw to Mn) was in the range of 2 to 3.

In an alternative embodiment, the hydrophobic polymer or monomers can be reacted with a poly(alkylene glycol) that is terminated with an amino function (available from Shearwater Polymers, Inc.) to form an amide linkage, which is in general stronger than an ester linkage.

Triblock or other types of block amphiphilic copolymers terminated with poly(alkylene glycol), and in particular, poly(ethylene glycol), can be prepared using the reactions described above, using a branched or other suitable poly(alkylene glycol) and protecting the terminal groups that are not to be reacted. Shearwater Polymers, Inc., provides a wide variety of poly(alkylene glycol) derivatives. Examples are the triblock PEG-PLGA-PEG.

Linear triblock amphiphilic copolymers such as PEG-PLGA-PEG can be prepared by refluxing the lactide, glycolide and polyethyleneglycol in toluene in the presence of stannous octoate. The triblock copolymer can also be prepared by reacting CH₃O(CH₂CH₂)ₙ—O-PLGA-OH with HO-PLGA.

In one embodiment, a multiblock amphiphilic copolymer is used and this may be prepared by reacting the terminal group of the hydrophobic polymeric block such as PLA or PLGA with a suitable polycarboxylic acid monomer, for example 1,3,5-benzenetricarboxylic acid, butane-1,1,4-tricarboxylic acid, tricarballylic acid (propane-1,2,3-tricarboxylic acid), and butane-1,2,3,4-tetracarboxylic acid, wherein the carboxylic acid groups not intended for reaction are protected by means known to those skilled in the art. The protecting groups are then removed, and the remaining carboxylic acid groups reacted with poly(alkylene glycol). In another alternative embodiment, a di, tri, or polyamine is similarly used as a branching agent.

Preferably the solution (I) and/or the precipitation agent (II) contains a stabilising agent for the organic compound. This stabilising agent can be, for example, the amphiphilic block polymer. Thus one of the solution (I) and the precipitation agent (II) may comprise the amphiphilic block polymer. In a preferred embodiment at least one of the solution (I) and the precipitation agent (II) comprises the amphiphilic block polymer and the other comprises a gelatine, especially a recombinant gelatine.

In addition, the wetting agent, when present, is preferably selected from the group consisting of sodium dodecylsulphate, Tween 80, Cremophor A25, Cremophor EL, Pluronic F68, Pluronic L62, Pluronic F88, Span 20, Tween 20, Cetomacrogol 1000, Sodium Lauryl Sulphate Pluronic F127, Brij 78, Klucel, Plasdone K90, Methocel E5, PEG, Triton X100, Witconol-14F and Enthos D70-30C. In case the particles that are precipitated according to the process of the present invention have to be used in a pharmaceutical application, it is preferred that the stabilising agent and the wetting agent are biocompatible.

According to an embodiment of the present invention, the wetting agent may be fed to the collecting vessel instead of the mixing chamber. According to another embodiment of the present invention, the stabilising agent and/or the wetting agent may be fed to both the collecting vessel and the mixing chamber.

The organic compound per se need not to be used in the process according to the present invention. It is possible to employ a precursor of the organic compound, wherein a precipitation agent is used that is capable of transforming this precursor into the organic compound per se. Consequently, according to this embodiment of the present invention, a precipitation agent is employed that is reactive with the precursor of the organic compound. This enables a substantially instantaneous chemical reaction between the precursor and the precipitation agent involving the formation of covalent or ionic bonds such as by protonation/deprotonation, by anion/cation exchange, by acid addition salt formation/liberation, redox reactions, addition reactions and the like. By the term “substantial instantaneous” a time is intended that is substantially shorter than the average residence time of (the precursor of) the organic compound in the mixing chamber.

It is important that the solution (I) of the organic compound is very well mixed with the precipitation agent (II) so that precipitation occurs in a controlled way in the part of the mixing chamber where the supersaturation allows for precipitation. By the continuous outflow of the precipitate of the organic compound and the liquid phase, a steady state is reached within the mixing chamber which can be maintained continuously. In general and preferably, the residence time in the mixing chamber is more than 0.0001 second and less than 5 seconds, preferably more than 0.001 second and less than 3 seconds. When the residence time is too Song, extremely fine grains once formed in the mixing chamber may grow to larger sizes and the average particle size distribution becomes undesirably wide. When the residence time is too short, too few nuclei may be formed. The optimum residence time will vary from one organic compound to another and may be optimised by simple trial and error.

The solution (I) and the precipitation agent (II) can be mixed in various manners, preferably so that a stable mixture of the solution (I) and the precipitation agent (II) in the closed mixing chamber is obtained. The solution (I) and the precipitation agent (II) are mixed by any mechanical stirring means, which can be driven in any way, for example by a drive shaft or by a rotating magnet. Preferably the mechanical stirring means is rotatable within the mixing chamber, for example it may comprise a rotatable blade. The blade may be in any form and have any aspect ratio, for example it may be in the form of a paddle where the ratio of its height to width are similar, or it may be in the form of disc, e.g. its height is very much smaller than its width. By width we mean the diametric distance from the central axis of rotation of the paddle to its outermost edge. It is preferred that the volume of the mechanical stirring means is at least 10% and not more than 99%, more preferably at least 15% and not more than 95% of the volume of the mixing chamber. Additionally, the mechanical stirring means may comprise a shaft and stirrer blade which may be rotated by the shaft. A preferred size of stirrer blade is at least 50%, more preferably at least 70%, especially 80% to 99% and a more especially 80% to 95% of the smallest diameter of the mixing chamber.

To assist with the mixing it is preferred that the precipitate of the organic compound and the liquid phase is discharged from the mixing chamber through an outlet which is towards the opposite end of the mixing chamber from the inlets and not directly line with the inlets. For example, the inlets may be positioned at the bottom part of the mixing chamber and the outlet(s) may be positioned at the top part of the mixing chamber. In one embodiment the inlets are below middle line of the chamber (e.g. below 30% height or 20% height). The outlet(s) may be above 70% height. In another embodiment, the outlet(s) is or are approximately at a right angle (e.g. 80° to 100° angle, especially 90° angle) relative to the flow of solution (I) and precipitation agent (II) through the inlets, in this way the liquids entering through the inlets do not immediately exit through the outlet without proper mixing.

In one embodiment the mixing chamber has more than one outlet.

The precipitate of the organic compound and the liquid phase are preferably discharged into a collecting vessel. The collecting vessel may comprise a second liquid phase comprising one or more of stabilisation agents, wetting agents, non-solvents, solvents or mixtures thereof

In another embodiment, ripening of the precipitate of the organic compound is performed in a collecting vessel until the preferred average particle size and/or average particle size distribution is achieved. This modification or ripening can be achieved by stirring the liquid phase and the precipitate in the collecting vessel. During modification or ripening, the average particle size may increase, but the average particle size distribution usually becomes narrower which is sometimes advantageous. Modification or ripening can be controlled by various parameters, e.g. temperature, pH or ionic strength. Consequently, according to this preferred embodiment, the process according to the present invention comprises a further step (e), wherein the precipitate of the organic compound and the liquid phase is discharged in a collecting vessel, wherein the precipitate of the organic compound is subjected to a ripening step.

In still another embodiment the precipitation agent comprises small particles of the compound to be precipitated. In this case larger particles can be obtained in a controlled way.

During an induction period of the precipitation process according to the present invention, the precipitation agent (II) is introduced with a continuous flow into the mixing chamber and may leaves the mixing chamber via the outlet to a collecting vessel. Subsequently, the solution (I) of the organic compound is introduced with a continuous flow into the mixing chamber which results in a supersaturation of the organic compound thereby initiating the formation of a precipitate and a liquid phase, in the liquid phase, the supersaturation may be reduced to such a level that essentially no precipitation will occur outside the mixing chamber. Since in this embodiment the solution (I) of the organic compound and the precipitation agent (II) are fed continuously, a continuous outflow of the precipitate and the liquid phase is eventually achieved. After the induction period, a steady state is reached in the mixing chamber meaning that basically the composition of the mixture within the mixing chamber is stable and essentially does not change over time. Additionally, the composition of the outflow of the precipitate and the liquid phase is stable and essentially does not change over time either.

The velocities of the inflow of solution (I) and precipitation agent (II) are not limited to high velocities. If multiple inlets are used, the velocity of one inflow may differ from the velocity of another inflow. However, in general the feed velocity of the inflow of the solution (I) and the precipitation agent (II) may be 0.01m/s, 0.1 m/s or 1 m/s. Even velocities of 10 m/s or more than 50 m/s can be used. The advantage of this inventive method is, however, that with relatively low feed velocities small particle precipitation can be achieved. Feed velocities in case of multiple inlets need not to be equal. In contrast, in impinging jet mixers it is important and in fact essential that these feed velocities match each other. The ratio of feed velocities of solution (I) and precipitation agent (II) can be 1:99 to 99:1. During the induction period, the effluent of the mixing chamber is collected until the composition of the effluent is essentially constant. As soon as a steady state is reached, the precipitate and the liquid phase are collected in a collecting vessel.

According to the invention, the organic compound to be precipitated, or precursors thereof are preferably dissolved in a solvent or solvent mixture as is mentioned above. The kind or nature of the precipitation agent (II) is dependent on the method of precipitation. In case of a solvent non-solvent precipitation, the precipitation agent is preferably a non-solvent, a mixture of non-solvents or a mixture of a non-solvent and a solvent, said mixture acting as a non-solvent. When a precipitation is caused by lowering the temperature, the precipitation agent is preferably a solvent or a solvent mixture having a temperature which initiates precipitation, in case of pH precipitation or ionic strength precipitation, the precipitation agent can be a solution having a pH or ionic strength, respectively, which initiates precipitation. In case of reaction precipitation, the precipitation agent will be a reactant which reacts with the precursor of the organic compound thereby inducing precipitation.

Söhnel and Garside (Precipitation, Basic Principles and Industrial Applications, Butterworth-Heinemann, 1992) have described the precipitation kinetics in a closed system, using classical nucleation theory. On page 113-114 they present the relation describing the critical nucleus size and the expected induction time. Classical nucleation theory primarily deals with the determination of the steady-state nucleation rate, J, i.e., the estimation of the number of supercritical clusters formed per unit time interval in a unit volume of a thermodynamically metastable system. In general, high values of J yield high numbers of particles and thus small particle sizes. Schmelzer and Slezov (Ch9: Theoretical Determination of the Number of Clusters Formed in Nucleation-Growth Processes, in: Aggregation Phenomena in Complex Systems, Ed.: J. Schmelzer, G. Ropke, R. Mahnke, Wiley-VCH, 1999) improved classical nucleation and growth theory by adopting less assumptions than classical theory does. For example, they dropped the assumption that growth of nuclei takes place one monomeric unit at a time. The supersaturation is one of the key parameters that dictate the nucleation and growth rate of solids during a precipitation. Nucleation theories have been successfully used extensively for salt precipitation but they have had limited success in predicting the particle size distribution of precipitated organic solids in a solvent anti-solvent precipitation.

In most practical batch applications, a steady-state can be established in a system only for a very short period of time. This is due to depletion of monomeric units from the system and therefore a drop in supersaturation. The actual supersaturation in a system after it has started precipitation is extremely hard to calculate or predict due to the aforementioned drop in monomer concentration and mixing inefficiencies. Baidyga et al. (J. Batelyga, W. Podgorska and R. Pohorecki, Chem. Eng. Sci., Vol. 50, No,8, pp 1281-1300, 1995.) reported work on BaSO₄ in a double-jet system in which turbulence models are combined with a complete nucleation and growth model (population balance) for a single vessel with a turbine agitator. The mathematical complexity of this work is huge.

In case of a continuous precipitator the balance of monomer feed, nucleation and growth of solid in the mixer and the outflow of supersaturation from the mixer cause the supersaturation to stabilize after some time.

In order to make the necessary simplifications to the nucleation rate calculations we treat the precipitation process as a plug-flow mixing process with perfect mixing at all times in the mixing chamber and we define a supersaturation ratio S₁₀ as follows (We neglect the formation of solid in the calculations):

$${S}_{10} = \frac{{C}_{10}}{{C}_{10 , e}}$$

wherein:

C₁₀ equals the concentration of solute at 10 seconds after addition start; and C_{10,e} equals the equilibrium solute concentration of solute at 10 seconds after addition start.

S₁₀ may be time-dependent if the flows, temperatures or concentrations are time-dependent. The 10 seconds allowed for start-up effects of unstabilised mixing chamber composition and temperature. Preferred experimental conditions are those that result in high values of S₁₀. Depending on the compound to be precipitated, S₁₀ values of more than 1.5, more than 2.5, more than 10 and even more have been found to be advantageous. For some compounds even a supersaturation value of 100 or more can prove advantageous.

The process according to the present invention is very suitable for precipitation of active pharmaceutical compounds into particles, possibly crystalline, with a small average size and a narrow particle size distribution. Small pharmaceutical particles are very suitable to be used in a medicament. Another advantage of the present invention is that the organic compound precipitates very purely.

The particles obtained by the process this invention can be of an amorphous nature or can be crystalline.

The organic compounds which can be precipitated according to the method of this invention, are preferably pharmaceutically active organic compounds, preferably selected from the group consisting of anabolic steroids, analeptics, analgesics, anaesthetics, antacids, anti-arrythmics, anti-asthmatics, antibiotics, anti-carcinogenics, anti-cancer drugs, anticoagulants, anticofonergics, anticonvulsants, antidepressants, antidiabetics, anti- diarrhoeal, anti-emetics, anti-epileptics, antifungals, antihelmintics, anti hemorrhoidals, antihistamines, antihormones, anti-hypertensives, anti-hypotensives, anti-inflammatories, antimuscarinics, antimycotics, antineoplastics, anti-obesity drugs, antiplaque agents, antiprotozoals, antipsychotics, antiseptics, anti-spasmotics, anti-thrombics, antitussives, antivirals, anxiolytics, astringents, beta-adrenergic receptor blocking drugs, bile acids, breath fresheners, bronchospasmolytic drugs, bronchodilators, calcium channel blockers, cardiac glycosides, contraceptives, corticosteroids, decongestants, diagnostics, digestives, diuretics, dopaminergics, electrolytes, emetics, expectorants, haemostatic drugs, hormones, hormone replacement therapy drugs, hypnotics, hypoglycaemic drugs, immunosuppressants, impotence drugs, laxatives, lipid regulators, mucolytics, muscle relaxants, non-steroidal anti-inflammatories, nutraceuticais, pain relievers, parasympatholytics, parasympathomimetics, prostaglandins, psychostimulants, psychotropics, sedatives, sex steroids, spasmolytics, steroids, stimulants, sulfonamides, sympathicolytics, sympathomimetics, sympathomimetics, thyreomimetics, thyreostatic drugs, vasodilators, vitamins, xanthines, and mixtures thereof. A particularly preferred organic compound is paclitaxel (also known as Taxol).

The size of the mixing chamber is dependent on the scale at which the precipitation is performed. On a small scale one typically would use a mixing chamber of volume 0.5 to 150 cm³ or 0.15-100 cm³, for medium scale a mixing chamber of 150 to 500 cm³ or 100-250 cm³ and for large scale mixing chamber of more than 500 cm³ to 1000 cm³ can be used. Preferably, the size of the mixing chamber is 1 cm³-1 dm³. As will be understood, the volume of the mixing chamber is volume without the mechanical stirring means being present. In a preferred embodiment the mixing chamber is a closed type mixing chamber.

Preferably at least one stirrer blade is positioned between the inlets such that it acts as a physical barrier between the incoming flows of the solution (I) precipitation agent (II). In this way the stirrer blade reduces the chance of precipitate formation at the inlets which could otherwise block these inlets. Instead the flows of the solution (I) precipitation agent (II) come into contact in a circumferential instead of ‘head-on’ manner.

A device which may be used to perform the process of the present invention is shown schematically in FIG. 1. The device according to this first preferred embodiment comprises a mechanical stirring means 1, a shaft 2, a mixing chamber 3, a mixing chamber wall 7, a first inlet 4 for feeding a solution (I) of the organic compound in a solvent, the inlet 4 being connected to the mixing chamber 3, a second inlet 5 for feeding a precipitating agent (II) to the mixing chamber 3, the inlet 5 being connected to the mixing chamber 3, and an outlet 6 for receiving a precipitate of the organic compound and a liquid phase, the outlet 6 being connected to the mixing chamber 3. For illustrative purposes, the mechanical stirring means 1 is depicted as a single stirrer blade, although more than one stirrer blade or other mechanical means which is rapidly movable relative to the chamber 3 may be used if desired. The positions as actually depicted in FIG. 1 for inlets 4 and 5 and for outlet 6 are also shown only for illustrative purposes. However, other positions of these inlets 4 and 5 and the outlet 6 are feasible and within the scope of the present invention. In particular, the positions of the inlets 4 and 5 and of the outlet 6 determine for a part the average residence time of the organic compound in the closed mixing chamber. In general, a mixing chamber has a bottom part and a top part. Furthermore, one can define a middle line through the mixing chamber dividing the mixing chamber in a bottom part and a top part. Furthermore, one can define the lowest bottom part as 0% height, the middle line as 50% height and the very top as 100% height. Using this general description of the mixing chamber, the inlets 4 and 5 should be connected at the bottom part of the mixing chamber that is below the middle line for example below 30% height or 20% height. The outlet 6 should be located at the upper part of the mixing chamber above the middle line, for example above 70% height. The inlets 4 and 5 may be diametrically opposed to each other. The inlets 4 and 5 may also be aligned in an essentially parallel fashion. The inlets 4 and 5 may also independently enter the mixing chamber via the lower bottom part. Likewise, outlet 6 is depicted in FIG. 1 as being positioned at the top of the mixing chamber 3. An advantage of this embodiment is that it does not require a bearing. Bearings can lead to contamination. Furthermore, by positioning outlet 6 at the top of the mixing chamber 3 helps by providing a more controlled outflow of the liquid including the precipitate.

The size of the mixing chamber 3 is dependent on the scale at which the precipitation is performed. On small scale one typically would use a mixing chamber of 0.5 to 150 cm³ or 0.15-100 cm³, for medium scale a mixing chamber of 150 to 500 cm³ or 100-250 cm³ and for large scale a mixing chamber of more than 500 cm³ to 1000 cm³ can be used, if desired. As will be understood, the volume of the mixing chamber is volume without the mechanical stirring means being present. Preferably, the size of the mixing chamber is 1 cm³-1 dm³.

The device is preferably provided with or may be connected to a collecting vessel. The collecting vessel preferably comprises a stirring means. Optionally, the mixing chamber may be surrounded by the collecting vessel. Alternatively, the mixing chamber may be positioned adjacent to or remote from the collecting vessel, dependent from the preference of the user. The device and/or the collecting vessel can be provided with a means to control temperature in e.g. mixing chamber and the collecting vessel, respectively. Such control means can for example be used to control the temperature of the solution (I), the precipitating agent (II), the closed type mixing chamber 3 and the supply tanks.

The device may comprise a supply tank (not shown) comprising the solution (I) of the organic compound and a supply tank (not shown) comprising the precipitation agent (II). The supply tanks may be connected to the mixing chamber by feed lines which can be, for example, hoses or fixed pipes. The transportation to the mixing chamber can be done with a continuous flow provided by a pump. The pump can be any pump known in the art as long as the pump can provide a stable flow during a prolonged period of time. Suitable pumps are for example plunger pumps, peristaltic pumps and the like.

The shape of the closed type mixing chamber can in principle be chosen freely and in case it is rotationally symmetric around a central axis, it can for example be specified by two identical surfaces, i.e. one top surface and one bottom surface, at a distance x from each other which surfaces may have any shape from rectangular to dodecagonal or circular with, when applicable, a minimum diameter of Dₘᵢₙ. For example, for a mixing chamber having a square shape, Dₘᵢₙ is the distance between opposite sides. In this embodiment, x can be larger than Dₘᵢₙ and alternatively, x can also be smaller than Dₘᵢₙ. In a further embodiment, the top surface and bottom surface need not to be identical, but one surface can be for example of a smaller size than the other.

A preferred device is shown in FIG. 2. This device is essentially the apparatus disclosed in U.S. Pat. No. 5,985,535, expressly incorporated by reference herein. In FIG. 2, the device comprises magnetically driven mechanical stirring means 1a and 1b, a mixing chamber 3 consisting of a chamber wall 7 having a central axis of rotation facing in top and bottom directions and seal plates 8 which function as tank walls seating top and bottom opening ends of the chamber wall 7. The chamber wall 7 and the seal plates 8 are preferably made of non-magnetic materials which are excellent in magnetic permeability if magnetically driven mechanical stirring means is employed which will be elucidated in more detail below, The stirring axes 2a and 2b are provided with outer magnets 9a, 9b and are disposed outside at the top and bottom ends of the mixing chamber 3 which are essentially opposite to each other. The outer magnets 9a, 9b are coupled to mechanical stirring means 1a, 1b inside the chamber via magnetic forces. Motors 10a and 10b drive the outer magnets 9a and 9b in converse directions. By this, mechanical stirring means 1a, 1b rotate in converse directions in the mixing chamber.

Further, in FIG. 2, the mixing chamber 3 is provided with a first inlet 4 for feeding a solution (I) of the organic compound in a solvent, the inlet 4 being connected to the mixing chamber 3, a second inlet 5 for feeding a precipitating agent (II) to the mixing chamber 3, the inlet 5 being connected to the mixing chamber 3, and a single outlet 6 for receiving a precipitate of the organic compound and a liquid phase, the outlet 6 being connected to the mixing chamber 3. Although inlets 4 and 5 are shown in a diametrically opposed fashion, they may also be aligned in an essentially parallel fashion. As the shape of the closed type mixing chamber 3, a cylindrical shape is often used, but rectangular, hexagonal and various other shapes may be used. Likewise, motors 10a, 10b driving outer magnets 9a, 9b via the axes 2a, 2b the mechanical stirring means 1a, 1b are shown as being disposed at the opposite top and bottom ends of the mixing chamber 3, but they may obviously be disposed at the opposite left and right sides, or may be disposed diagonally, depending on the shape of the mixing chamber. Additionally, the mixing chamber 3 may comprise more pairs of conversely rotating mechanical stirring means.

In another embodiment of the device according to FIG. 2, an odd number of magnetically driven mechanical stirring means may be used, e.g. one, three or five magnetically driven mechanical stirring means. Furthermore, the use of pair wise oriented mechanical stirring means in combination with a single stirring means may lead to even more efficient stirring.

A preferred process comprises the following steps, e.g. using a device as shown in FIG. 2:
(I) feeding a flow (i) comprising a solution (I) comprising the organic compound and a solvent via a first inlet to a closed type mixing chamber and contacting flow (i) with a flow (ii) comprising a precipitating agent (II) fed simultaneously with flow (i) via a second inlet to the mixing chamber thereby forming a flow (iii) comprising a precipitate of the organic compound and a liquid phase; and
(II) discharging flow (iii) comprising the precipitate of the organic compound and the liquid phase from the mixing chamber, preferably in a geometric direction cocurrent with the direction by which flow (i) comprising the solution of the organic compound is fed to the mixing chamber, via a single outlet or via more than one outlet.

The term “cocurrent direction” is to be understood that the direction of flow (iii) is not counter current to the direction of flow (i). The term “cocurrent direction” is more in particular to be understood as that the angle defined by the axis of flow (i) and the axis of flow (iii) varies from 90° to 180°.

In this preferred process, it is further preferred that flow (ii) comprising the precipitating agent (II) is fed to the mixing chamber in a direction essentially diametrically opposed to the direction by which the flow (i) comprising the solution (I) comprising the organic compound is fed to closed type mixing chamber.

In another preferred embodiment the device according to FIG. 3 is used. In FIG. 3, the device comprises a mechanical stirring means 1, a mixing chamber 3 consisting of a chamber wall 7 having a central axis of rotation facing in top and bottom directions. Stirring means 1 is disposed preferably in the centre of the mixing chamber 3, occupies a large % of the volume of the chamber and can be driven preferably directly via a stirrer axis 2 and a motor (not shown). The inlets 4 and 5 are preferably essentially perpendicular to each other. However, the positions of inlets 4 and 5 are interchangeable, that is that inlet 4 may enter the mixing chamber 3 via the bottom thereof whereas inlet 5 may enter the mixing chamber 3 via a sidewall. Alternatively, inlet 5 may enter the mixing chamber 3 via the bottom thereof whereas inlet 4 enters the mixing chamber 3 via a sidewall, ft is also possible that both inlets 4 and 5 enter through the side wall, in which the angle in a horizontal plane between the inlets can have any value, but is preferably between 90° and 180°. In this embodiment the stirrer axis or shaft 2 is positioned within the outlet 6 of the mixing chamber 3. It is further possible that both inlets 4 and 5 enter via the bottom part of the mixing chamber 3. In a preferred embodiment, inlet 5 via which the anti solvent enters the mixing chamber is placed at the bottom. In this embodiment unwanted precipitation at the inlet into the reaction chamber is prevented.

Additionally, in one embodiment it is also highly preferred that the volume of the stirring means 1 is at least 10% (e.g. more than 80%) and not more than 99%, preferably not more than 95%, of the volume of the mixing chamber 3. Hence, this preferred embodiment of the invention uses a precipitation device comprising a stirring means 1 comprising an axis or shaft 2, a mixing chamber 3 comprising a chamber wall 7 having a central axis of rotation facing in top and bottom directions, an inlet 4 and an inlet 5 that are preferably essentially perpendicular to each other, and an outlet 6 in which axis or shaft 2 of stirring means 1 is positioned.

The device according to the embodiment of FIG. 3 may be constructed from moveable parts as is shown in FIGS. 3A and 3B illustrating a top view of this embodiment of the device. Here, the mixing chamber 3 is formed by two moveable chamber parts 11 that are rotatable around hinges 12. The movable chamber parts 11 interlock around mechanical stirring means 1 (a stirrer blade in the form of a rotatable disc) driven by shaft 2.

According to the invention, a preferred process comprises the following steps, e.g. using a device as shown in FIG. 3:
(I) feeding a flow (i) comprising a solution (I) comprising the organic compound and a solvent via a first inlet to a mixing chamber and contacting flow (i) with a flow (ii) comprising a precipitating agent (II) fed simultaneously with flow (i) via a second inlet to the mixing chamber thereby forming a flow (iii) comprising a precipitate of the organic compound and a liquid phase; and
(II) discharging flow (iii) comprising the precipitate of the organic compound and the liquid phase from the mixing chamber in a geometric direction essentially perpendicular to either the direction by which flow (i) comprising the solution of the organic compound is fed to the mixing chamber or the direction by which flow (ii) comprising the precipitating agent (II) is fed to the mixing chamber.

Alternatively, step (II) may also comprise discharging flow (iii) comprising the precipitate of the organic compound and the liquid phase from the mixing chamber in a geometric direction essentially cocurrent with either the direction by which flow (i) comprising the solution of the organic compound is fed to the mixing chamber or the direction by which flow (ii) comprising the precipitating agent (II) is fed to the mixing chamber or with both if both inlets enter the mixing chamber via its bottom part.

Another device which may be used to perform the process of the present invention is shown in FIG. 4. Also this embodiment may be constructed from moveable parts as is shown in FIGS. 3A and 3B.

In FIG. 4, the device comprises mechanical stirring means 1a, 1b in disc form, mixing chamber 3 consisting of compartments and is the free area between the chamber wall 7 and the stirring means 1a, 1b and shaft 2. Also in this embodiment the stirrer axis or shaft 2 is positioned within the single outlet 6 of the mixing chamber 3. The inlets 4 and 5 are preferably essentially perpendicular to each other. However, also in this embodiment the positions of inlets 4 and 5 are interchangeable and also in this embodiment inlets 4 and 5 may enter the mixing chamber through the side walls or via the bottom part of the mixing chamber. In a preferred embodiment the precipitation agent (II) enters via the bottom part of the mixing chamber.

Additionally, in this embodiment at least, it is also highly preferred that the volume of the mechanical stirring means 1, which in this case have a disc shape 1a, 1b, is at least 10% (e.g. at least 80%) and not more than 99%, preferably not more than 95%, of the volume of the mixing chamber 3. In the embodiment shown the stirring axis comprises two disks 1a, 1b and the mixing chamber 3 comprises compartments made by separating wall 13. A mixing chamber with one disk as mechanical stirring means can also be used, while also mixing chambers having three or more compartments, each compartment being provided with a disk as mechanical stirring means attached to one single axis, can be used. Hence, in this preferred embodiment the device comprises at least one, more preferably two, three, four or more mechanical stirring means in the form of disks being driven by shaft 2, a mixing chamber 3 consisting of a chamber wall 7 having a central axis of rotation facing in top and bottom directions, said mixing chamber 3 comprising an inlet 4 and an inlet 5 that are preferably essentially perpendicular to each other, and an outlet 6 in which is positioned shaft 2 driving stirring means 1. Optionally, mixing chamber 3 may be divided in compartments by one or more separating walls 13. Within the scope of this embodiment are devices comprising more than one stirring disk as mechanical stirring means in a mixing chamber that is not separated into one or more compartments by one or more separating walls, as well as devices comprising more than one stirring disk as mechanical stirring means and a mixing chamber separated into several compartments by one or more separating walls. Obviously, if the device comprises only a single stirring disk as mechanical stirring means, it will generally not comprise a separating wall, so that the mixing chamber comprises only one compartment.

Also the device according to FIG. 4 allows for a process comprising the following steps:
(I) feeding a flow (i) comprising a solution (I) comprising the organic compound and a solvent via a first inlet to a mixing chamber and contacting flow (i) with a flow (ii) comprising a precipitating agent (II) fed simultaneously with flow (i) via a second inlet to the mixing chamber thereby forming a flow (iii) comprising a precipitate of the organic compound and a liquid phase; and
(II) discharging flow (iii) comprising the precipitate of the organic compound and the liquid phase from the mixing chamber in a geometric direction essentially perpendicular to either the direction by which flow (i) comprising the solution of the organic compound is fed to the mixing chamber or the direction by which flow (ii) comprising the precipitating agent (II) is fed to the mixing chamber.

Like the embodiment of the FIG. 3, step (II) may also comprise discharging flow (iii) comprising the precipitate of the organic compound and the liquid phase from the mixing chamber in a geometric direction essentially cocurrent with either the direction by which flow (i) comprising the solution of the organic compound is fed to the mixing chamber or the direction by which flow (ii) comprising the precipitating agent (II) is fed to the mixing chamber or with both if both inlets enter the mixing chamber via its bottom part.

Preferably, all parts of the mixing chamber that are in contact with the mixture in the mixing chamber are coated with a layer of a material that prevents adhering, fouling, incrustation and the like. Preferred materials are those having moisture absorption according to ASTM D 570 at a relative humidity of 50% and a temperature of 23° C. of less than 1%. Suitable examples of such materials include fluorinated alkene polymers and copolymers, e.g. polytetrafuoroethylene, and polyacetals, e.g. polyoxymethylene.

At the start of the nucleation, nuclei are usually surrounded by over-saturated fluid. When two or more of these particles stay in contact for too long, they will be “cemented” together to form an agglomerate. Furthermore, unlike inorganic particles in aqueous media, organic particles are usually not electrically charged and therefore these organic particles do not have a strong electrostatic repulsive mechanism. In the present invention, the drag/shear forces in the mixing chamber imposed on the nuclei by the fluid motion may prevent the particles from agglomerating. In one embodiment of this invention, excessive turbulence is used to reduce the inter-particle contact times to values that do not allow agglomeration to any material extent while the surrounding fluid is still over-saturated.

In the present invention it was found that a preferred diameter of the mechanical stirring means is at least 50% and more preferably at least 70% and most preferably between 80 and 99% of the smallest diameter of the mixing chamber. Very good results were obtained with a mechanical stirring means which had a diameter of around 90% to 95% of the smallest diameter of the mixing chamber. In another embodiment, very good results were obtained with a mechanical stirring means which had a diameter of 80% to 90% of the smallest diameter of the mixing chamber.

In the present invention, when opposite mechanical stirring means are driven in the mixing chamber (i.e. the shafts rotate in opposite directions), it is preferable to rotate the mechanical stirring means at high speed to obtain a high mixing efficiency. The rotation speed is preferably 1,000 rpm or more, more preferably 3,000 rpm or more, and especially 5,000 rpm or more. A pair of conversely rotating stirring means may be rotated at the same rotating speed or at different rotating speeds. In case of a mechanical stirring means which is symmetrical around an axis, the stirrer speed should be more than 500 rpm, for example 1,000 rpm or 5,000 or even 10,000 rpm. Nowadays, mechanical stirrers are commercially available having a stirrer speed of 20,000 rpm and even more. In general, the higher the stirring speed the better the mixing and therefore there is no particular upper limit for the stirring speed.

The residence time of the organic compound in the mixing chamber can be varied amongst others by changing various parameters, e.g. the inflow of the solution (I) of the organic compound, the inflow of the precipitation agent (II), the choice of the type, e.g. shape and size, of the mechanical stirring means, intensity of mixing and positions of the inlets and the single outlet. A too short residence time in the mixing chamber is undesirable as it may result in uncontrolled nucleation outside the mixing chamber. A too long residence time in the mixing chamber is also undesirable as it may result in excessive agglomeration and growth. Solvent and non-solvent, together with for example the temperature, can be selected to control the rate of the nucleation. The nucleation time can for example be from 10⁻⁹ to 10⁻² seconds. The mixing is therefore an important factor, because reduced mixing efficiencies at these very high nucleation speeds can cause undesirable agglomeration.

Also for compounds not having such a fast nucleation time, the residence times in the mixing chamber should not be too long, because the efficiency of the precipitation process will be lowered. Furthermore, a long residence time may result in a wide average particle size distribution and larger particles. In practice, the mixing chamber residence time preferably does not exceed 3 seconds and is below 1 second. In case nucleation proceeds slowly, e.g. from 10⁻³ until 10⁻⁶seconds, the conditions are preferably chosen such that the residence time is more than 0.1 but below 5 seconds, more preferably below 3 seconds and even more preferably below 1 second.

The residence time t may be calculated as follows:

t=v/(a+b)

wherein:

v is the volume of the mixing space of a mixing vessel (cm³);

a is the addition flow of an organic compound solvent solution (cm³/sec); and

b is the addition flow of the precipitation-agent (cm³/sec).

Preferably the precipitated organic compound arising from the process has an average particle size of less than 1 micron, more preferably less than 700 nm, especially less than 500 nm, more especially less than 200 nm, Preferably the precipitated organic compound has a unimodal particle size distribution.

If desired the process may also include the step of drying the precipitated organic compound, for example using a spray drier. Preferably drying of the precipitated organic compound is begun within 10 minutes of performing step (c), more preferably within 5 minutes, especially within 2 minutes and more especially within 1 minute of performing step (c). in this way any subsequent growth of particle size is reduced or avoided altogether.

The process of the present invention may be performed on any scale and steps (a) to (d) may be performed in a continuous manner. In this way large quantities of the desired particulate organic compound may be prepared, including on the industrial scale. There is no need to include jets in the process which have to be carefully aligned. The conditions may be tailored to give small particles which can be isolated and redispersed without difficulty.

The process is particularly useful for preparing pharmaceutical actives in a particulate form, it may also be used to provide particles of other organic compounds, for example agrochemicals, colorants, cosmetics and the like.

Preferably the precipitated organic compound is in particulate form and has a D50 of less than 500 nm, more preferably less than 400 nm, especially less than 300 nm, more especially less than 200 nm. The D50 may be measured by techniques known in the art, for example by Laser diffraction using the method according to ISO 13320-1, e.g. using a Malvern Mastersizer 2000 particle size analyser.

In another aspect the present invention also provides a process for the manufacture of medicament comprising performing the process of the present invention wherein the organic compound is a pharmaceutically active compound.

Preferably this process further comprises the step of mixing the product of the process with a pharmaceutically acceptable carrier or excipient to give the medicament.

The identity of the carrier or excipient is not crucial provided it is pharmaceutically acceptable. Examples of such carriers and excipients include the diluents, additives, fillers, lubricants and binders commonly used in the pharmaceutical industry.

In a preferred aspect the medicament is in the form of a tablet, troche, powder, syrup, patch, liposome, injectable dispersion, suspension, capsule, cream, ointment or aerosol.

Thus, medicaments intended for oral use may contain, for example, one or more colouring, sweetening, flavouring and/or preservative agents in addition to the product of the presently claimed process (the product of the presently claimed process often being abbreviated herein as simply as “the active ingredient”).

Suitable pharmaceutically acceptable carriers and excipients for a tablet or troche formulation include, for example, inert diluents such as lactose, sodium carbonate, calcium phosphate or calcium carbonate, granulating and disintegrating agents such as corn starch or algenic acid, binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient either dissolved or in particulate form together with one or more suspending agents, such as sodium carboxymethylcellulose, methylcellulose, hydroxypropylmethylcellulose, sodium alginate, polyvinylpyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain alphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate), anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient, optionally together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The medicaments of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The medicaments may also be in the form of a sterile injectable aqueous or oily suspension, which may be formulated according to known procedures using one or more of the appropriate dispersing or wetting agents and suspensing agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the return to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Medicaments for administration by insufflation may be in the form of particles made by the presently claimed process, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Medicaments for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

If desired the process may further comprise the step of sterilising the precipitated pharmaceutically active organic compound. The object of the sterilisation is to kill any undesirable bacteria which may cause harm to a patient, particularly if their immune system has been compromised. Typical sterilisation methods include irradiation, heating and treatment with a biocide.

The pharmaceutically active compound referred to in the above further aspects of the present invention may be any of the pharmaceutically active organic compounds mentioned earlier in this specification, especially paclitaxel or a cyclosporin (e.g. cyclosporin A).

Also the invention provides a medicament obtained by the process of the present invention.

Also the invention provides a method for the treatment of a human or animal comprising administration of a medicament obtained by the process of the present invention. Also the invention provides use of a pharmaceutically active organic compound obtained by the process of the present invention for the manufacture of a medicament for the treatment of cancer.

The invention is now illustrated by the following non-limiting examples in which all parts and percentages are by weight unless otherwise specified.

In all examples the chemicals used were:

The following chemicals were obtained from Sigma-Aldrich Co., Zwijndrecht,

The Netherlands:

Paclitaxel from taxus brevifolia, ≧95% (HPLC),

Pregnenolone, ≧98%,

Fenofibrate, ≧99% powder,

Cyclosporin A, BioChemika, ≧98.5% (TLC),

Tetrahydrofuran (THF) biotech grade ≧99.9%, inhibitor-free,

Citric acid, USP grade,

D-Mannitol, USP grade,

The MPEG-PLA block copolymers.

The anhydrous ethanol 100% DAB, PH.EUR. was obtained from Boom B. V., Meppel, The Netherlands,

Fish gelatin 150 kDa was obtained from Norland Products Inc., Cranbury, USA,

Hydrolysed fish gelatin 4.2 kDa was obtained from Nitta Gelatin Inc., Japan,

The water used was purified by demineralization and filtration techniques on-site.

### EXAMPLE 1

In this example the organic compound, fenofibrate, was precipitated in a device of the general type described in U.S. Pat. No. 5,985,535, using an amphiphilic block copolymer.

### (A) Preparation of Solution (I)

An ethanolic solution was prepared containing fenofibrate (20 g/l) and poly(ethylene glycol)-block-polylactide methyl ether (PEG Mₙ 750, PLA Mₙ 1000, (4.4 g/l); commercially available from Sigma Aldrich). The temperature of the solution was adjusted to 293K.

### (B) Preparation of Precipitation Agent (II)

A precipitation agent was prepared comprising an anti-solvent was prepared consisting of water and non-hydrolysed non-gelling fish gelatine, molecular weight average 150 kDa (4 g/l). The temperature of this anti-solvent was adjusted to 293K.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solvent solution (I) was 10 cm³/min and the feed rate for the precipitation agent (II) was 110 cm³/min. The stirrer blades had diameters of 83% of the chamber diameter and were operated at 6,000 RPM in opposite directions. Turbidity was observed immediately after introduction of the solvent solution and precipitation agent.

The total batch addition time to make 100 cm³ of product was 50 seconds. The resultant particles were discharged from the chamber through the outlet port and collected.

The particle size distribution of the resultant particles was measured using a Malvern Mastersizer 2000. The particles were found to have a unimodal particle size distribution and the average particle size was in the nanometer range. The D50 of the particles was 111 nm. The D90 of the particles was 206 nm.

### EXAMPLE 2

### (A) Preparation of Solution (I)

A solution was prepared comprising tetrahydrofuran and paclitaxel (10 g/l) and poly(ethylene glycol)-methyl ether block-polylactide (PEG average Mn 5000, PLA average Mn 5000) (10 g/l) at 20° C.

(B) Preparation of Precipitation agent (II)

The precipitation agent (II) was pure water at 0° C.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solvent solution was 15 cm³/min and the feed rate for the precipitation agent (II) was 105 cm³/min. The ratio of solvent solution to precipitation agent (II) was 20:100. The stirrer blades had diameters of 83% of the chamber diameter and the stirrers were operated at 6000 RPM in opposite directions.

The initial particle size (D50) of the resultant particles was approximately 260 nm.

### EXAMPLE 3

### (A) Preparation of Solution (I)

A solution was prepared comprising tetrahydrofuran and paclitaxel (10 g/l) and poly(ethylene glycol)-methyl ether block-polylactide (PEG average Mn 350, PLA average Mn 1000) (10 g/l) at 20° C.

### (B) Preparation of Precipitation Agent (II)

The precipitation agent (II) was pure water at 0° C.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solution (I) was 15 cm³/min and the feed rate for the precipitation agent (II) was 105 cm³/min. The ratio of solution (I) to precipitation agent (II) was 15:105. The stirrer blades had diameters of 83% of the chamber diameter and the stirrers and were operated at 6000 RPM in opposite directions.

The initial particle size D(50) of the resultant particles was approximately 123 nm.

### EXAMPLE 4

### (A) Preparation of Solution (I)

A solution was prepared comprising tetrahydrofuran and paclitaxel (10 g/l) and poly(ethylene glycol)-methyl ether block-polylactide (PEG average Mn 750, PLA average Mn 1000) (10 g/l) at 20° C.

### (8) Preparation of Precipitation Agent (II)

The precipitation agent (II) was a pure water at 0° C.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solution (I) was 15 cm³/min and the feed rate for the precipitation agent (II) was 105 cm³/min. The ratio of solvent solution to precipitation agent (II) was 15:105. The stirrer blades had diameters of 83% of the chamber diameter and the stirrers were operated at 6000 RPM in opposite directions.

The initial particle size of the resultant particles was below 115 nm.

### EXAMPLE 5

### (A) Preparation of Solution (I)

A solution was prepared comprising tetrahydrofuran and cyclosporin A (10 g/l) and poly(ethylene glycol)-methyl ether block-polylactide (PEG average Mn 750, PLA average Mn 1000) (10 g/l) at 20° C.

### (B) Preparation of Precipitation Agent (II)

The precipitation agent (II) was a 1wt % solution of citric acid in pure water at 0° C.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solution (I) was 15 cm³/min and the feed rate for the precipitation agent (II) was 105 cm³/min. The ratio of solution (I) to precipitation agent (II) was 15:105. The stirrer blades had diameters of 83% of the chamber diameter and the stirrers were operated at 6000RPM in opposite directions.

The initial particle size of the resultant particles was approximately 132 nm.

### EXAMPLE 6

Amphiphilic Polymer which is not an Amphiphilic Block Copolymer

In this example the organic compound, pregnenolone, was precipitated in a device of the general type described in U.S. Pat. No. 5,985,535, using an amphiphilic copolymer which was not a block copolymer.

The method of Example 1 was repeated except that in place of solution (I) there was used pregnenolone in ethanol (34 g/l) at 50° C. and the precipitation agent was water containing 4 wt % of hydrolysed non-gelling fish gelatine, molecular weight 4.2 kDA. The total batch addition time to make 100 cm³ of product was 50 seconds. The resultant particles were discharged from the chamber through the outlet port and collected.

The particle size distribution was measured with a Malvern Mastersizer 2000. The particles had a bimodal particle size distribution. The D50 of the particles was 1.36 μm. The D90 of the particles was 4.58 μm.

### EXAMPLE 7

### (A) Preparation of Solution (I)

A solution was prepared comprising tetrahydrofuran and paclitaxel (10 g/l) and poly(ethylene glycol)-methyl ether block-polylactide (PEG average Mn 750, PLA average Mn 1000) (10 g/l) at 20° C.

### (8) Preparation of Precipitation Agent (II)

The precipitation agent (II) was pure water containing citric acid (1 wt %) and mannitol (5 wt %) at 0° C.

### (C) The Process

The solution (I) and the precipitation agent (II) were fed simultaneously into the mixing chamber of a device of the general type shown in U.S. Pat. No. 5,985,535, FIG. 1, having a cylindrical chamber with an internal volume of 1.5 cm³, two spaced inlets, a pair of magnetically driven stirrer blades as mechanical stirring means and one outlet. The feed rate for the solution (I) was 15 cm³/min and the feed rate for the precipitation agent (II) was 105 cm³/min. The ratio of solution (I) to precipitation agent (II) was 15:105. The stirrer blades had diameters of 83% of the chamber diameter and the stirrers were operated at 6000 RPM in opposite directions.

The D50 reported by the Mastersizer was 118 nm.

### Comparative Example 1

### Continuously Stirred Tank—No Amphiphilic Polymer—No Simultaneous Addition

A solution of pregnenolone in ethanol (34 g/l) was added over 45 seconds, with stirring, to a tank containing pure water as precipitation agent (1500 cm³). The rate of addition was 1000 cm³/min. The stirrer rotational speed was 750 rpm. Turbidity was observed immediately after the addition started.

Pregnenolone was precipitated and its particle size was analysed using a Malvern Mastersizer 2000. The precipitate was found to have a wide particle size distribution, including many particles of 10 μm edge length or more. The D50 of the particles was 14.59 μm. The D90 of the particles was 36.22 μm.

### Comparative Example 2

### Chamber—No Amphiphilic Polymer

The method of Example 1 was repeated except that in place of solution (I) there was used pregnenolone in ethanol (34 g/l) and water was used as the precipitation agent. The solvent solution and the precipitation agent were fed into the chamber at 275K. The total batch addition time to make 100 cm³ of product was 50 seconds. The resultant particles were discharged from the chamber through the outlet port and collected.

The particle size distribution was measured with a Malvern Mastersizer 2000. The particles had a narrower particle size distribution than Comparative Example 1. and the average particle size was in the nanometre range. The D50 of the particles was 9.17 μm. The D90 of the particles was 18.72 μm.

### SUMMARY OF RESULTS

| Example: | 1 | Comparative 1 | Comparative 2 | 2 |
|---|---|---|---|---|
| Method | As | Stirred tank | As | As |
| | claimed | | claimed | claimed |
| Amphiphilic | Yes | No | No | Yes |
| polymer | | | | |
| used? | | | | |
| Particle size | Unimodal | Very wide | Narrower than | bi modal |
| distribution | | distribution | Comparative 1 | |
| D50 (nm) | 111 | 14,590 | 9,170 | 260 |
| D90 (nm) | 206 | 36,220 | 18,720 | 483 |

| Example: | 3 | 4 | 5 | 6 | 7 |
|---|---|---|---|---|---|
| Method | As | As | As | As | As |
| | claimed | claimed | claimed | claimed | claimed |
| Amphiphilic | Yes | Yes | Yes | Yes (but not | Yes |
| polymer | | | | a block | |
| used? | | | | copolymer) | |
| Particle size | tri- | Mono- | Mono- | Bimodal | Mono- |
| distribution | modal | modal | modal | | modal |
| D50 (nm) | 123 | 115 | 132 | 1,360 | 118 |
| D90 (nm) | 219 | 175 | 238 | 4,580 | 184 |

Footnotes:
1) D50 and D90 measured immediately after the process.

1-28. (canceled)

29. A process for the precipitation of an organic compound, wherein:
(a) a solution (I) of the organic compound in a solvent is introduced via a first inlet into a mixing chamber;
(b) a precipitation agent (II) is introduced, simultaneously with step (a), via a second inlet into the of mixing chamber;
(c) the solution (I) of the organic compound and the precipitation agent (II) are mixed thereby forming a precipitate of the organic compound and a liquid phase; and
(d) the precipitate of the organic compound and the liquid phase is discharged from the mixing chamber via one or more outlets;
wherein step (c) is performed using a mechanical stirring means in the presence of an amphiphilic polymer.

30. A process according to claim 29, wherein the amphiphilic polymer is an amphiphilic block copolymer comprising a PEG Mn 250-5000 block and/or a PEG Mn 250-5000(C₁₋₄-alkyl) ether block.

31. A process according to claim 30, wherein the amphiphilic polymer is an amphiphilic block copolymer comprising a PLA Mn 250-5000 block.

32. A process according to claim 31, wherein the amphiphilic polymer is an amphiphilic diblock or triblock copolymer.

33. A process according to claim 31, wherein the volume of the stirring means is at least 10% of the volume of the mixing chamber.

34. A process according to claim 31, wherein the solution (I) and the precipitation agent (ii) are miscible.

35. A process according to claim 30, wherein the inlets are connected at below 30% height of the mixing chamber and the outlet(s) are located above 70% height of the mixing chamber.

36. A process according to claim 31, wherein the inlets are connected at below 30% height of the mixing chamber and the outlet(s) are located above 70% height of the mixing chamber.

37. A process according to claim 31, wherein the residence time of the organic compound in the mixing chamber is longer than 0.1 milliseconds and shorter than 5 seconds.

38. A process according to claim 31, wherein the mixing in step (c) is performed by a plurality of mechanical stirring means rotating in opposite directions.

39. A process according to claim 30, wherein the organic compound is a pharmaceutically active organic compound.

40. A process according to claim 39, wherein the organic compound is paclitaxel or a cyclosporin.

41. A process according to claim 29 wherein:
(a) the amphiphilic polymer comprises one or more PEG and/or PEG ether blocks and one or more PLA blocks;
(b) the said mixing is performed by a plurality of mechanical stirring means rotating in opposite directions;
(c) the solvent comprises an organic solvent;
(d) the solution (I) and/or the precipitation agent (II) contains the amphiphilic block copolymer;
(e) the residence time of the organic compound in the mixing chamber is longer than 0.1 milliseconds and shorter than 5 seconds; and
(f) the solution (I) and the precipitation agent (II) are miscible.

42. A process according to claim 41, wherein the inlets are connected at below 30% height of the mixing chamber and the outlet(s) are located above 70% height of the mixing chamber.
